(19) United States
(12) Patent Application Publication
(10) Pub. No.: US 2023/0260659 A1
(43) Pub. Date: Aug. 17, 2023

(54) METHOD FOR A PREDICTIVE PROGNOSIS OF THE ONSET OF A CARDIOVASCULAR DISEASE

(71) Applicant: Allelica S.R.L., Rome (IT)

(72) Inventors: Giordano BOTTA', Rome (IT); Paolo DI DOMENICO, Rome (IT); Alessandro BOLLI, Rome (IT)

(21) Appl. No.: 18/005,469

(22) PCT Filed: Jul. 14, 2021
(86) PCT No.: PCT/IB2021/056339

§ 371 Date: Jan. 13, 2023

(30) Foreign Application Priority Data
Jul. 16, 2020 (IT) 102020000017326

(51) Int. Cl. G16H 50/30; G16B 40/20; G16H 10/60; G16H 50/20

(52) U.S. Cl. CPC G16H 50/30; G16B 40/20; G16H 10/60; G16H 50/20

(57) ABSTRACT

A method for a predictive prognosis of an individual's cardiovascular disease onset calculates a cardiovascular disease risk value based on the individual's polygenic risk score and low-density lipoprotein cholesterol level, and risk parameters representative of: an onset risk induced by: the polygenic risk score, the low-density lipoprotein cholesterol level, and interaction of polygenic risk score and low-density lipoprotein cholesterol level. The method determines and provides at least one of: an individualized risk value of onset of cardiovascular disease in an individual; and/or an individualized target value of low-density lipoprotein cholesterol level to cause a risk corresponding to that of an individual having: an average polygenic risk score, and/or an individualized equivalent value of low-density lipoprotein cholesterol level to cause, an average polygenic risk score and the same individualized value of low-density lipoprotein cholesterol level. An individual's polygenic risk score value is calculated based on genetic data.

### TECHNOLOGICAL BACKGROUND OF THE INVENTION

### Field of Application

The present invention relates to a method for a predictive prognosis of the onset of a cardiovascular disease.

Therefore, the general technical field of the present invention is that of predictive methods, performed by means of electronic computation, used in the medical field to support predictive prognoses.

More in particular, the present invention also relates to a method for calculating the correct LDL cholesterol level for the genetic component of an individual and for identifying the LDL cholesterol levels required for the risk reduction based on the genetic component of the individual.

### Description of the Prior Art

Cardiovascular diseases are the main cause of death in the world, and are caused by a combination of genetic and lifestyle factors. Cardiovascular diseases are preventable by means of actions aimed at improving lifestyle or by means of the timely prescription and execution of pharmacological therapies aimed at identified high-risk subjects.

This makes the early identification of high-risk individuals absolutely important.

In this regard, several predictive models have been developed recently, and are thus known, for estimating the risk of cardiovascular disease, both in absolute terms (for example, risk of incurring a cardiovascular disease in the next ten years) and in relative terms (for example, risk compared to that of the average population).

Such known models include, for example, “Q-Risk” (in this regard, consider the article by J. Hippisley-Cox et al. “Performance of the QRISK cardiovascular risk prediction algorithm in an independent UK sample of patients from general practice: a validation study” https://heart.bmi.com/content/94/1/34.full), or “Score Risk” (in this regard, consider for example the information accessible at the Internet link https://www.escardio.org/Education/Practice-Tools/CVD-prevention-toolbox/SCORE-Risk-Charts), or again “Framingham” (consider, for example, the article by P. W. F. Wilson et al. “Prediction of Coronary Heart Disease Using Risk Factor Categories” https://www.ahajournals.org/doi/10.1161/01.CIR.97.18.1837).

However, these models are not completely satisfactory, and in particular they are relatively ineffective, since they fail to identify most cases in the category considered high risk.

The most widely used known models currently take into account several risk factors, including age, gender, smoking, the presence of previous co-morbidities, such as diabetes or hypertension, and also cholesterol levels, in particular the low-density lipoprotein cholesterol level LDL-C and the high-density lipoprotein cholesterol level HDL.

Very recently, a new genetic risk factor has emerged as important, different from traditional risk factors; this new genetic risk factor is measurable by a parameter called polygenic risk score (PRS), which proved to be suitable to identify people at risk even in the absence of other already known risk factors.

Therefore, taking into account the genetic risk factor, for example through the PRS, potentially allows an improvement in the predictive capacity of the existing models.

However, it is not easy to find a methodology which allows the genetic risk factor to be appropriately and effectively combined with other risk factors, and the need remains strongly felt to have prognostic methods capable of providing more reliable, accurate and precise results, especially with regard to the correlation of risk due to genetic factors and risk related to physiological factors such as cholesterol level.

Furthermore, studies carried out at the population level show that the risk of developing coronary heart disease in people with a high genetic risk may be equal to or even higher than that of individuals with lower LDL-C levels, but who also have a lower genetic risk.

Therefore, in order to estimate more reliably the risk of onset of cardiovascular disease, and avoid that results based on classic risk factors (such as cholesterol level), may even be misleading.

Finally, there is a further need to translate the quantification of the risk factor into further prognostic results, adapted to support an effective and personalized diagnosis and therapeutic prescription.

### SUMMARY OF THE INVENTION

It is the object of the present invention to provide a method for a predictive prognosis of the onset of a cardiovascular disease, which allows to obviate at least partially the drawbacks mentioned above with reference to the known art, and to meet the aforementioned needs particularly felt in the technical field considered. Such an object is achieved by a method according to claim 1.

Further embodiments of the method are defined by claims 2-16.

Moreover, it is the object of the invention to provide a device adapted to perform a predictive prognosis of the onset of a cardiovascular disease based on the aforesaid method. Such an object is achieved by a device according to claim 17.

Moreover, it is the object of the invention to provide a method for providing a clinical evaluation or for deriving a therapeutic intervention based on the aforementioned prognosis method. Such an object is achieved by a method according to claim 18 or claim 19.

### BRIEF DESCRIPTION OF THE DRAWINGS

Further features and advantages of the method according to the invention will become apparent from the following description of preferred embodiments, given by way of non-limiting example, with reference to the accompanying drawings, in which:

FIG. 1 shows a table, and a related graph, showing how the genetic factor considerably affects the overall risk level, with the same cholesterol level;

FIG. 2 shows an example of presentation a table of the results which are obtainable by the method of the present invention;

FIG. 3 is a table showing the level of correlation between polygenic risk and traditional risk factors;

FIG. 4 is a table depicting known intervention guidelines, available to physicians, to decide on a possible curative intervention based on the cholesterol level LDL-C and non-genetic risk factors calculated according to known models;

FIG. 5 is a table depicting intervention guidelines, made available to physicians by virtue of the method of the present invention, to decide on a possible curative intervention taking into account not only the cholesterol level LDL-C (and any other non-genetic risk factors) but also genetic risk factors calculated by means of the polygenic risk score.

### DETAILED DESCRIPTION

A method for a predictive prognosis of the onset of a cardiovascular disease in an individual is described.

Such a method firstly comprises the step of calculating at least one risk value R, representative of the risk for the onset of cardiovascular disease in the individual based on an operational value of the individual's polygenic risk score PRS and an operational value of the individual's low-density lipoprotein cholesterol level LDL-C, and further based on a first risk parameter HR₁ representative of a risk of the onset of cardiovascular disease induced by the polygenic risk score PRS, on a second risk parameter HR₂ representative of a risk of the onset of cardiovascular disease induced by the low-density lipoprotein cholesterol level LDL-C, and on a third risk parameter HR₃ representative of a risk of the onset of cardiovascular disease induced by the interaction of the polygenic risk score PRS and the low-density lipoprotein cholesterol level LDL-C.

The aforementioned first risk parameter HR₁, second risk parameter HR₂ and third risk parameter HR₃ are calculated based on a pre-trained model and/or algorithm, to which the operational value of the aforesaid polygenic risk score PRS and the operational value of low-density lipoprotein cholesterol level LDL-C are provided in input.

The method then includes determining and providing, based on the aforesaid at least one calculated risk value R, at least one of the following prognostic results: an individualized risk value Rₚ of onset of cardiovascular disease in an individual to be examined; and/or an “individualized target value” VOI of low-density lipoprotein cholesterol level LDL-CT such as to cause, for an individual having a given polygenic risk score PRSₚ, a risk corresponding to that of an individual having an average polygenic risk score PRSₘ, and/or an “individualized equivalent value” VEI of low-density lipoprotein cholesterol level LDL-C_{E} such as to cause, for an individual having a given polygenic risk score PRSₚ and a given individualized low-density lipoprotein cholesterol level LDL-Cₚ, a risk corresponding to that of an individual having an average polygenic risk score PRSₘ and the same individualized low-density lipoprotein cholesterol level value LDL-Cₚ of the individual to be examined.

The method further comprises the steps of accessing the genetic data of the individual to be examined, comprising the individual's single nucleotide polymorphisms SNPs, and calculating a polygenic risk score value PRSₚ of the individual, based on the aforesaid genetic data of the individual.

In the case where the result of the prognosis is the individualized risk value Rₚ, the method comprises the further step of acquiring or accessing data comprising the individual's low-density lipoprotein cholesterol level LDL-Cₚ.

Furthermore, the step of calculating at least one risk value R comprises calculating a risk value R considering as the operational value of polygenic risk score PRS the value PRSₚ of the individual's polygenic risk score and as the operational value of the low-density lipoprotein cholesterol level LDL-C the individual's low-density lipoprotein cholesterol level LDL-Cₚ. The individualized risk value Rₚ of onset of cardiovascular disease corresponds to the aforesaid calculated risk value R.

In the case where the result of the prognosis is the individualized target value VOI of low-density lipoprotein cholesterol level LDL-C_{T}, the method comprises the further step of defining a plurality of sample values of low-density lipoprotein cholesterol level LDL-Cₙ.

Furthermore, the step of calculating at least one risk value R comprises, for each sample value of low-density lipoprotein cholesterol level LDL-Cₙ, calculating a respective plurality of sample risk values Rₙ in which the polygenic risk score is the individual's polygenic risk score PRSₚ and a respective plurality of average risk values Rₘₙ in which the polygenic risk score is a preset average polygenic risk score PRSₘ.

The step of determining an individualized target value VOI of low-density lipoprotein cholesterol level LDL-C_{T} comprises determining the individualized target value VOI of low-density lipoprotein cholesterol level LDL-C_{T} as the value of the plurality of sample values of low-density lipoprotein cholesterol level LDL-Cₖ for which the respective sample risk value Rₖ is equal to the respective average risk value Rₘₖ.

In the case where the result of the prognosis is the individualized equivalent value VEI of low-density lipoprotein cholesterol level LDL-C_{E}, the method comprises the further steps of acquiring or accessing data comprising the individual's low-density lipoprotein cholesterol level LDL-Cₚ, and defining a plurality of sample values of low-density lipoprotein cholesterol level LDL-Cₙ.

Furthermore, the step of calculating at least one risk value R comprises calculating an individualized risk value Rₚ considering as the operational value of polygenic risk score PRS the individual's polygenic risk score value PRSₚ and as the operational value of low-density lipoprotein cholesterol level LDL-C the individual's low-density lipoprotein cholesterol level LDL-Cₚ.

The step of calculating at least one risk value R further comprises calculating, for each sample value of low-density lipoprotein cholesterol level LDL-Cₙ, a respective plurality of average risk values Rₘₙ in which the polygenic risk score is a preset average polygenic risk score PRSₘ.

Lastly, the step of determining an individualized equivalent value VEI of low-density lipoprotein cholesterol level LDL-C_{E} comprises determining the individualized equivalent value VEI of low-density lipoprotein cholesterol level LDL-C_{E} as the value of the plurality of sample values of low-density lipoprotein cholesterol level LDL-Cₙ for which the respective average risk value Rₘₙ is equal to the individual's individualized risk value Rₚ.

As noted above, the method allows to achieve different types of prognostic results, and in particular the determination of one or more of the aforesaid individualized risk value Rₚ of onset of cardiovascular disease, and/or “individualized target value” VOI of low-density lipoprotein cholesterol level LDL-C_{T}, and/or “individualized equivalent value” VEI of low-density lipoprotein cholesterol LDL-C_{E}.

According to different implementation options, the method includes providing one, or two, or all three of the above results, in any combination.

In particular, according to an embodiment of the method, the aforesaid prognostic results comprise the individualized risk value Rₚ of onset of cardiovascular disease in an individual to be examined and further at least one of the aforesaid individualized target value VOI of low-density lipoprotein cholesterol level LDL-C_{T} and individualized equivalent value VEI of low-density lipoprotein cholesterol level LDL-C_{E}.

According to another embodiment of the method, the aforementioned prognostic results comprise the individualized target value VOI of low-density lipoprotein cholesterol level LDL-C_{T} and the individualized equivalent value VEI of low-density lipoprotein cholesterol level LDL-C_{E}.

In accordance with an embodiment of the method, the aforementioned pre-trained model and/or algorithm (by means of which the risk parameters HR₁, HR₂ and HR₃ are calculated) comprises an algorithm trained in a preliminary training step, which includes training the algorithm by means of “machine learning” and/or artificial intelligence techniques, based on experimental data referring to individuals for which both the respective individual values of the polygenic risk score PRS and the operational values of the low-density lipoprotein cholesterol level LDL-C, and the onset or non-onset of cardiovascular disease is also known.

From what has been illustrated above, it is clear that the method of determining the risk parameters (preparatory to the subsequent calculation of the risk factors) does not depend on the set of experimental data used; the method can be applied by those skilled in the art, based on the teachings provided in this description, using an available experimental dataset, of the type indicated above.

According to an implementation option, the pre-trained model or algorithm comprises a regression model, which uses as covariates the polygenic risk score PRS, low-density lipoprotein cholesterol level LDL-C, combination of polygenic risk score and low-density lipoprotein cholesterol level (PRS×LDL-C).

In such a case, the preliminary training step comprises training the regression model using as a reference dataset a first dataset of a population of individuals of which PRS and CDC-L are known, and for which the outcomes of onset of cardiovascular disease are known, for a first subset of individuals (the so-called “cases”), or for which the non-onset of cardiovascular disease is known, after a predetermined period of time, for a second subset of individuals (the so-called “controls”).

In accordance with an implementation option, the pre-trained regression model is used to derive the aforementioned first risk parameter HR₁, second risk parameter HR₂ and third risk parameter HR₃, and the preliminary training step comprises training the regression model with reference to the aforesaid first risk parameter HR₁, second risk parameter HR₂ and third risk parameter HR₃ based on the reference dataset, already mentioned above, using as a dependent variable the onset or non-onset, obtainable from known data, of cardiovascular disease.

In accordance with an implementation option, the pre-trained regression model is used to calculate said at least one risk value R. In this case, the preliminary training step comprises training the regression model with reference to the aforesaid at least one risk value R based on the aforesaid reference dataset, using as a dependent variable the onset or non-onset, obtainable from known data, of cardiovascular disease.

In accordance with an implementation option, the regression model is a Cox proportional hazards regression model.

In accordance with an embodiment, the method is adapted to perform a predictive prognosis which takes into account one or more further risk factors.

In such a case, the aforesaid step of calculating at least one risk value R comprises calculating the at least one risk value R also based on an operational value of each of one or more further covariates COVₓ associated with a respective one or more of the aforesaid further risk factors.

Furthermore, the pre-trained model and/or algorithm is configured to calculate the aforesaid first risk parameter HR₁, second risk parameter HR₂ and third risk parameter HR₃ also based on the operational values of each of the aforesaid one or more further covariates COVₓ.

In such a case, the method further comprises accessing data of the individual to be examined, comprising individual values COVₓₚ of each of said one or more further covariates associated with the one or more further risk factors.

As part of the determination of the individualized target value VOI of low-density lipoprotein cholesterol level LDL-C_{T}, the step of calculating at least one risk value R comprises, for each sample value of low-density lipoprotein cholesterol level LDL-Cₙ, calculating a respective plurality of sample risk values Rₙ in which the polygenic risk score is the polygenic risk score of the individual PRSₚ and the value of each of the further covariates associated with a respective risk factor corresponds to, or is representative of, the individual value of the respective covariate associated with the risk factor COVₓₚ found in the individual.

The calculating step further comprises calculating a respective plurality of average risk values Rₘₙ in which the polygenic risk score is a preset average polygenic risk score PRSₘ, and the value of each of the further covariates associated with a respective risk factor corresponds to, or is representative of, the individual value of the respective covariate associated with the risk factor COVₓₚ found in the individual.

As part of the determination of the individualized equivalent value VEI of low-density lipoprotein cholesterol level LDL-C_{E}, the step of calculating at least one risk value R comprises calculating an individualized risk value Rₚ considering as the operational value of polygenic risk score PRS the value PRSₚ of the individual's polygenic risk score and as the low-density lipoprotein cholesterol level LDL-C the individual's low-density lipoprotein cholesterol level LDL-Cₚ, and as the operational value of each of the aforesaid further covariates associated with a respective risk factor a value corresponding to, representative of, the individual value of the respective covariate COVₓₚ found in the individual.

The calculating step further comprises calculating, for each sample value of low-density lipoprotein cholesterol level LDL-Cₙ, a respective plurality of average risk values Rₘₙ in which the polygenic risk score is a preset average polygenic risk score PRSₘ, and the value of each of the further covariates associated with a respective risk factor corresponds to, or is representative of, the individual value of the respective covariate associated with the risk factor (COVₓₚ) found in the individual.

As already indicated, in a possible implementation option, the value of each of the further covariates corresponds to the individual value of the respective covariate associated with the respective risk factor COVₓₚ found in the individual.

In other possible implementation options, the value of each of the further covariates is related by means of a predetermined known function (e.g., the logarithm) to the individual value of the respective covariate associated with the respective risk factor COVₓₚ found in the individual.

In accordance with an implementation example of the method, in which the pre-trained model and/or algorithm comprises a regression model, which uses as covariates, in addition to the polygenic risk score PRS, low-density lipoprotein cholesterol level LDL-C, combination of polygenic risk score and low-density lipoprotein cholesterol level (PRS×LDL-C), also the aforesaid further variables which encode the further risk factors COVₓ.

In such a case, the preliminary training step comprises training the regression model using as reference datasets a second dataset from a population of individuals of which, in addition to PRS and LDL-C, the individual values of each of such further covariates (COVₓ) are known as well.

In accordance with an embodiment of the method, in which the individualized target value VOI of low-density lipoprotein cholesterol level LDL-C_{T} is determined, the method further comprises the following steps:
determining in which percentile the individualized polygenic risk score PRSₚ falls within a distribution of values of polygenic risk score PRS of individuals of a known reference population;
performing a plurality of simulations Sₙ, each using the pre-trained model and/or algorithm, in which each simulation Sᵢ is performed on an individual with the same polygenic risk score value as the individual examined, and/or having the same values of further covariates COVₓ associated with other risk factors of the individual under examination, and on a respective sample value low-density lipoprotein cholesterol level LDL-Cₙ, among a set of increasing values of low-density lipoprotein cholesterol level LDL-Cₙ which vary from the minimum to the maximum of the values present in the reference population on which the pre-trained model was trained.

Each simulation Sᵢ determines a risk level Rᵢ|LDLᵢ, PRSₚ×LDLᵢ, PRSₚ, COV_{x,p} corresponding to a respective sample value of low-density lipoprotein cholesterol level LDL-Cᵢ.

Each of the risk levels Rᵢ is compared with the risk calculated on an individual with the same features of the individual to be examined, but having an average genetic risk, i.e., with an average PRS, PRSₘ.

In accordance with another embodiment of the method, in which the individualized equivalent value VEI of low-density lipoprotein cholesterol level LDL-C_{E} is determined, the method further comprises the following steps:
performing a plurality of simulations Sₙ, each using the pre-trained model and/or algorithm, in which each simulation Sᵢ is performed on an individual having the same values of covariates associated with other risk factors COVₓ as the individual in question, but having an average polygenic risk score value PRSₘ, and on a respective sample value of low-density lipoprotein cholesterol level LDL-Cₙ, among a set of increasing values of low-density lipoprotein cholesterol level LDL-Cₙ which vary from the minimum to the maximum of the values present in the reference population on which the pre-trained model was trained.

Each simulation Sᵢ determines a risk level (Rᵢ|LDLᵢ, PRSₘ×LDLᵢ, PRSₘ, COV_{x,p}) corresponding to a respective sample value of low-density lipoprotein cholesterol level LDL-Cᵢ, to obtain the low-density lipoprotein cholesterol level LDL-C which confers the same final risk of developing cardiovascular disease for an individual with the same features as the individual to be examined, but an average polygenic risk score PRSₘ.

According to an embodiment of the method, the risk value R is calculated based on a sum of products related to different relevant variables (i.e., relevant covariates), comprising at least the aforesaid polygenic risk score PRS, low-density lipoprotein cholesterol level LDL-C, combination of polygenic risk score and low-density lipoprotein cholesterol level PRS×LDL-C, in which each product involves the multiplication of the first risk parameter HR₁, second risk parameter HR₂ and third risk parameter HR₃ for the difference between the operational value and the average value of the variables, respectively, polygenic risk score PRS, low-density lipoprotein cholesterol level LDL-C, combination of polygenic risk score and low-density lipoprotein cholesterol level (PRS×LDL-C).

According to an implementation option, in accordance with the Cox regression model, the risk value is calculated based on the following formula:

R(t|x)=b₀(t)exp(Σᵢ₌₁ⁿbᵢ×(xᵢ−x̅ᵢ))

where:
R (t|x) is the risk value;
b₀(t) is a baseline risk value, the same for all individuals;
bᵢ are the partial risk parameters (often referred to as “partial hazard”), each associated with a respective variable or covariate;

for each variable, the parameter bᵢ is estimated in the model and represents the risk increase factor or risk decrease factor due to the difference between the value of the dependent variable of the individual (for example PRS and LDL) and the value of that same variable in the population average; the parameters bᵢ thus pre-calculated are then applied to the individual to obtain his/her personalized risk value R to develop a coronary artery disease at a certain age or after a certain period of time; it should be noted that, according to an implementation option, for the covariates “polygenic risk score PRS”, “low-density lipoprotein cholesterol level LDL-C”, “combination of polygenic risk score and low-density lipoprotein cholesterol level PRS×LDL-C”, the respective parameter bᵢ corresponds respectively to the logarithms of the first risk parameter HR₁, second risk parameter HR₂ and third risk parameter HR₃;
xᵢ are the considered variables (covariates), comprising at least the aforesaid covariates “polygenic risk score PRS”, “low-density lipoprotein cholesterol level LDL-C”, “combination of polygenic risk score and low-density lipoprotein cholesterol level PRS×LDL-C” and further comprising, optionally, in some embodiments of the method, the further covariates COVₓ associated with further risk factors;
x̅ₗ are the average values of the respective covariates;
the sum Σᵢ₌₂ⁿbᵢ×(xᵢ−x̅ᵢ) is the logarithm of the partial risk (“log-partial hazard”), and represents the change in the risk of the individual due to the effect of all the independent variables included in the model.

According to an embodiment of the method, the determined or calculated risk value is a relative risk value, compared to an average risk referring to a population.

In accordance with another embodiment of the method, the determined or calculated risk value is an absolute risk value, related to the individual, and representative of the individual's risk of developing cardiovascular disease within a certain period of time from when the method is applied to examine the individual, or at a given age.

According to an implementation example, the aforesaid time period corresponds to 10 years.

According to an implementation option, in which the absolute risk value is representative of the individual's risk of developing cardiovascular disease within a given age, the training step of the pre-trained model and/or algorithm is performed, using as duration variable the age at which the subject possibly developed coronary artery disease (individual of the “Cases” subpopulation in the training dataset) or alternatively, in the case where the individual has never developed it (individual of the “Controls” subpopulation) the age of the individual at the end of the study, i.e., the initial age plus a predetermined number of years (for example 10).

According to another implementation option, in which the absolute risk value is representative of the individual's risk of developing cardiovascular disease within a certain period of time from when the method is applied to examine the individual, the training step of the pre-trained model and/or algorithm is performed using as duration variable the time elapsed from the start of the study to the possible occurrence of cardiovascular disease for individuals in the “cases” subpopulation of the training dataset, or alternatively the full duration of the study for the “controls” subpopulation of the training dataset.

According to an implementation option of the method, the risk value R is representative of a probability density function PDF of onset of cardiovascular disease with respect to time.

According to another implementation option of the method, the risk value R is representative of a cumulative probability density function CDF of onset of cardiovascular disease over time.

In various application examples of the method, it is used for a predictive prognosis of different cardiovascular diseases.

According to a preferred application example, the method is used for a predictive prognosis of a coronary artery disease or CAD.

In accordance with an embodiment of the method, the step of calculating a value PRSₚ of the individual's polygenic risk score, based on the genetic data of the individual, comprises identifying, in the individual's genetic data, a customized subset SI₁ of single nucleotide polymorphisms SNPs which also belong to a set I₁, in which each of the single nucleotide polymorphisms SNPs of said predetermined set I₁ comprises a single nucleotide polymorphism SNP identifier, and is associated with a respective relevance parameter βᵢ; and then calculating the individual's polygenic risk score value PRSₚ, based on said customized subset SI₁ of single nucleotide polymorphisms SNPs and the respective relevance parameters βᵢ.

According to an implementation option, the aforesaid single nucleotide polymorphism SNP identifier comprises a genetic variant address and an active allele present in the genetic variant address; the aforesaid customized subset SI₁ comprises single nucleotide polymorphisms SNPs, in which the active allele is traced as present in the respective genetic variant address, in the individual's genetic data, and is associated with a respective allelic dosage; the aforesaid step of calculating a value PRSₚ of individualized polygenic risk score comprises adding the value of all the first relevance parameters βᵢ associated with all the respective single nucleotide polymorphisms SNPs of the customized subset SI₁, each multiplied by the respective allelic dosage of the active allele.

It should be noted that, in the embodiment mentioned above, the processing of the biological data acquired from the individual allows to obtain a summary score, i.e., the aforesaid PRS, of the genetic risk consisting of the sum of the risk value associated with each SNP.

According to an implementation option, the determination of the predetermined set I1 of single nucleotide polymorphisms SNPs and the calculation of the relevance parameters βi are performed in a preliminary training step, comprising the training of at least one algorithm using “machine learning” and/or artificial intelligence techniques, based on known data.

According to another implementation option, the aforementioned predetermined set I₁ of single nucleotide polymorphisms SNPs and the relevance parameters βᵢ are taken from known tables.

For example, in an implementation option of the method (to which the results reported below refer) the association between SNP and relevance parameter is performed using a PRS table published in 2019 in the scientific article by Bolli et. al. https://www.biorxiv.org/content/10.1101/763722v2 “Software as a Service for the Genomic Prediction of Complex Diseases”.

With reference to the multiplication by the “allelic dosage”, such an operation involves multiplying the relevance parameter, associated with a certain SNP, by the number of effective alleles present in the respective genetic variant address.

According to the most common implementations, the “allelic dosage”, therefore the number by which the relevance parameter is multiplied, is 0 or 1 or 2.

It should be noted that the term “effective allele” (used most often by technical experts in the field) can also be referred to as “risk allele”.

With reference to the aforesaid criterion for identifying the most relevant SNPs, the method includes various implementation variants, for example:

(i) identifying as the most relevant SNPs those which are associated with the highest relevance parameter values β₁; or

(ii) testing different PRSs calculated on different SNPs, validating the PRS on known populations and choosing those which result in a better predictivity using as a metric the “sensitivity”, i.e., the ability to identify people affected by the disease as at risk, or the “specificity”, i.e., the ability to identify unaffected people as not at risk; or

(iii) applying AUC-ROC (area under the receiver operator characteristic curve-ability to distinguish cases from controls) methodologies known per se.

According to an embodiment of the method, the preliminary training step comprises building the first predetermined set I₁ of single nucleotide polymorphisms SNPs by means of a selection of relevant single nucleotide polymorphisms SNPs carried out through the following steps: identifying single nucleotide polymorphisms SNPs statistically associated with the risk of onset of cardiovascular disease through a genetic association study (several genetic risk association studies of cardiovascular disease based on SNPs are known in this regard), in which each of such identified single nucleotide polymorphisms SNPs is associated with a respective known initial relevance parameter β.

Optionally, optimal values of relevance parameters β₁ are then identified to optimize the predictive effectiveness of the first polygenic risk score PRS value.

The optimization of the predictive efficacy of the first polygenic risk score PRS value is carried out from the aforesaid identified single nucleotide polymorphisms SNPs and respective initial known relevance parameters (β), through the single or combined use of predictive algorithms known per se, such as Clumping+Thresholding, LD-Pred, Stacked Clumping+Thresholding (SCT).

Each of such predictive algorithms is trained based on a known dataset (previously also referred to as a training dataset, according to a common term in the field) containing genetic data of single nucleotide polymorphisms SNPs of individuals for which the non-onset or the onset age and course of the cardiovascular disease considered is known.

The training then is completed with the steps of defining the aforesaid first predetermined set I₁ of single nucleotide polymorphisms SNPs based on the identified single nucleotide polymorphisms SNPs; and defining as respective first relevance parameters β₁ the aforesaid respective optimal values of identified relevance parameters β₁. As already noted, according to some implementation options, the method is based on known tables of single nucleotide polymorphisms SNPs and relevance parameters βᵢ which weigh the effect of the SNPs with respect to the risk of onset of cardiovascular disease.

However, it should be noted that, even in this case, the scope of the method goes well beyond the individual known tables, because it is applicable to different populations.

With reference to the latter aspect, it is known that a score such as the PRS has a higher predictive value if calculated on an individual with the same ancestral derivation as the individuals based on which the PRS was derived, i.e., the individuals of the database with which the method was trained.

A difference between populations of different ancestrality lies, for example, in the difference in the Linkage Disequilibrium maps, which affects the determination of the causal SNPs.

With regard to the known dataset containing genetic data of single nucleotide polymorphisms SNPs of individuals in relation to the onset of coronary artery disease (CAD), one example of the method, actually implemented, uses the “UK BioBank” database, which is the largest genetic database in the world, within the project: “Validating genomic trait prediction algorithms for commercial use with the UK BioBank resource”.

The “UK BioBank” database (https://www.ukbiobank.ac.uk/) contains the genetic information of over 500,000 volunteers, providing clinical information. Hence, in the example shown here, the PRS has a higher predictive validity for Caucasians.

However, it is again underlined that the method described here is agnostic with respect to the population analyzed and is applicable to data related to any type of population/ancestrality, as long as the algorithms used are trained based on a known database related to such a population/ancestrality.

It should also be noted that, by applying the method starting from a population of different ethnicity, specific predictors would be obtained for the population on which the PRS was derived, i.e., different tables and different weight parameters.

As already described above, in an implementation option, the method uses the parameters PRS, LDL-C and the interaction thereof, possibly together with other risk factors (covariates), to predict within a regression model (for example, proportional Cox risks) pre-trained to obtain the absolute and relative risk of developing a coronary artery disease consists in training a model using as the reference dataset a population of which the traditional risk factors and the PRS thereof calculated on the same basis as the PRS calculated for the individual are known.

A first particularly innovative aspect of the method described here is the ability to take into account the genetic risk factor by means of the polygenic risk score PRS.

In fact, from studies conducted by the Applicant, it was found that the polygenic risk is largely orthogonal with respect to traditional risk factors.

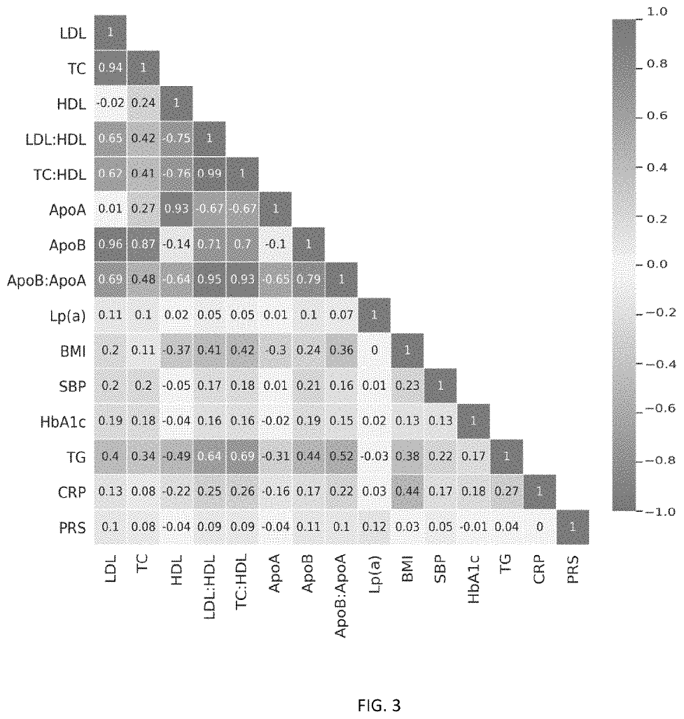

This is illustrated in the table reported in FIG. 3, which shows the level of correlation between polygenic risk and traditional risk factors. The number within each square represents the correlation between one risk factor and another. If such a number were equal to 1, then the correlation between the two risk factors would be complete and as one risk factor grew the other risk factor would also increase correspondingly. If there is a complete correlation between two risk factors, the presence within the model of both risk factors would not add any further information. The predictive capacity of the model would be unchanged because one variable (therefore one factor) could be directly replaced by the other one.

In contrast, taking into account a risk factor orthogonal to the others (for example, the PRS) provides further and significant information (not derivable from other risk factors) and therefore allows improving the predictive capacity of the model.

The table in FIG. 3 shows how the polygenic risk score is “orthogonal” with respect to other risk factors, and is therefore able to identify people at risk of developing a cardiovascular problem which would not have been identified using only the other factors without taking PRS into account.

It should be noted that the risk factors in FIG. 3 are the following (the acronyms shown in the figure and the definition in English, universally known and used): TC: total cholesterol, HDL: high-density cholesterol, LDL-C: low-density cholesterol, LDL-C:HDL: low-density over high-density cholesterol ratio, TC:HDL: low-density over high-density cholesterol ratio, ApoA: Apolipoprotein A, ApoB: Apolipoprotein B, Ln(a): Lipoprotein a, BMI: Body Mass Index, SBP: Systolic Blood Pressure, HbA1C: Glycated hemoglobin, TG: Triglycerides, CRP: C-reactive protein, PRS: SCT-I Polygenic risk score.

A further innovative aspect of the method described here is the ability to take into account the interaction between genetic risk factor by means of the polygenic risk score PRS and the cholesterol value LDL-C.

In an implementation option of the method, such an interaction is explicitly represented in the model by means of an autonomous covariate (LDL-C×PRS).

According to another implementation option of the method, such an interaction is not explicitly represented in the model by means of an autonomous covariate, but is implicitly present and active in the model, based on the presence of the covariates LDL-C and PRS, and acts as a sort of “force” which is intrinsic in the results of the calculation of the relative risk.

For example, for the cases exemplified in this description, as already noted above, the population data of UK Biobank (about 500,000 individuals) were used, the risk factors of which and the genetic data thereof from which the PRS was computed are known.

Further illustrations of the method according to the invention and of the results obtainable therewith will be provided below by way of example.

Table 1 below shows a result obtained by carrying out the method described above of the present invention, and in particular an extract of a regression carried out with a Cox proportional hazards model on the known population of the UK Biobank database, using the presence or absence of a coronary artery disease as a dependent variable.

TABLE 1

| | R (95% CI) | P value |
|---|---|---|
| PRS | 1.45 (1.39-1.53) | <0.005 |
| LDL-C | 1.39 (1.32-1.45) | <0.005 |
| PRS × LDL-C | 1.06 (1.02-1.11) | <0.005 |

Column R shows the average value of the calculated risk level R (and the standard deviation range in brackets), with reference to the parameter PRS, considered separately, the parameter LDL-C, considered separately, and a factor PRS×LDL-C which takes into account the correlation between the two aforesaid factors.

The P-Value column provides the P-Value, which, as known in inferential statistics, in particular in tests of hypothesis verification, is the probability of obtaining results equal to or less likely than that observed during the test, assuming the null hypothesis to be true. In other words, the P-value helps to understand whether the difference between the result observed and the hypothesized result is due to the randomness introduced by sampling, or whether such a difference is statistically significant. P-Value values below 0.05 (as in the case of table 1) indicate that the results obtained are statistically significant.

Table 1 shows that, in addition to the further risk induced per se by the genetic risk factor PRS and the cholesterol level LDL-C (respectively 1.40 and 1.44 for standard deviation), there is a risk induced by a multiplicative combination of these two factors which suggests that a high LDL-C level is more harmful (i.e., results in a higher risk) for people with a high genetic risk with respect to people with an average genetic risk.

Table 1 provides significant additional information and advances with regard to already known evidence which already indicated the significant effect of genetic risk (associated with the polygenic risk score parameter) on the overall risk, in addition to the risk due to cholesterol level, but which did not show the correlation between the two risk factors.

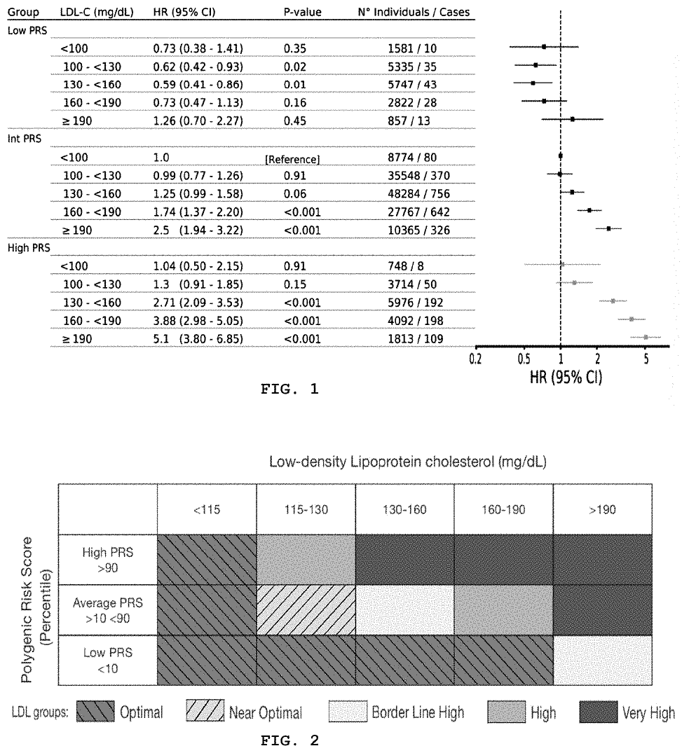

FIG. 2 depicts a summary table showing the overall risk of the onset of coronary artery disease (CAD), based on the combination of the variables polygenic risk score PRS and cholesterol level LDL-C.

The rows of the table refer to the high, medium, or low PRS value depending on whether it is >90 percentile, or between 10 and 90 percentile, or less than 10 percentile (parameterized on an average population).

The columns refer to different LDL-C level ranges that can be found in the individual under examination.

Each box is assigned a risk group. In the example shown, there are 5 risk groups: Optimal, Near Optimal, Border Line High, High, Very High.

As can be seen in the table, LDL-C cholesterol increases the risk of developing coronary artery disease in different ways depending on polygenic risk.

In people with high polygenic risk, LDL-C levels considered average by the guidelines (130-160 mg/dl) confer a higher risk than that conferred by LDL-C levels considered high risk (>190 mg/dl) in people with average polygenic risk.

Furthermore, people with high polygenic risk (>90th percentile of PRS) have a risk which is no higher than the average with optimal LDL-C levels (<130 mg/dL). This suggests that, by obtaining optimal LDL-C cholesterol levels, it is possible to reduce the risk to that of the average population.

Therefore, the method described herein offers the advantage of providing physicians with a new tool able to customize the target LDL-C cholesterol level to be achieved for each individual based not only on traditional risk factors, but also on the genetic risk factor.

The invention further comprises a prognostic device adapted to carry out a predictive prognosis of the onset of a cardiovascular disease.

Said device comprises: acquisition means for acquiring an amount of blood of an individual and determining the low-density lipoprotein cholesterol level LDL-C of the individual based on an examination of the amount of blood acquired; electronic interface means, adapted to receive in input a calculated value of the individual's polygenic risk score PRS; processing means, configured to receive the aforementioned low-density lipoprotein cholesterol level LDL-C and polygenic risk score PRS value of the individual, and configured to carry out a method for a predictive prognosis of the onset of a cardiovascular disease according to any of the previously described embodiments.

The aforesaid acquisition and determination means, electronic interface means and processing means are included in a single portable device.

According to an embodiment of the device, said acquisition and determination means comprise a medical measuring device capable of taking a drop of blood from the individual and obtaining the LDL-C cholesterol level of the individual.

According to an implementation option, such a medical measuring device is made using “finger” technology, which is known per se (consider for example the article by Parin Parikh et al. “Clinical Utility of a Fingerstick Technology to Identify Individuals With Abnormal Blood Lipids and High-Sensitivity C-Reactive Protein Levels” https://www.ncbi.nlm.nih.gov/pmc/articles/PMC2750040/), which allows obtaining an autonomous device for calculating the LDL value at low costs.

According to an implementation option of the device, inside the medical measuring device of the “finger stick” type, the electronic interface (for example, keyboard or “touch-screen” type) and the electronic processor in which the software program executing the algorithm(s) for calculating the prognosis method described above is also advantageously integrated.

According to an implementation option, the algorithm is integrated inside the device which carries out the LDL cholesterol test with the addition of the necessary instructions to record, during the setup step (as for example occurs for the time), the insertion by the user of the calculated polygenic risk level thereof and capable of providing in output the result of processing the risk prognosis and the aforesaid “individualized target value” of cholesterol LDL-C and “individualized equivalent value” of cholesterol LDL-C.

Such an integration is possible, for example, on all devices mounting operating systems which can interpret information in any programming language (from machine language to Java) by virtue of the possibility of translating the corresponding LDL level calculation model into executable binary code.

Thereby, the device according to the invention, described herein, can be seen as a medical measuring device which, by taking a drop of blood and recording the PRS thereof, outputs one or more of the aforesaid results, and allows the user to know in real time what the corresponding LDL cholesterol level is based on the polygenic risk level thereof entered as input in the configuration step.

The invention further comprises a method for providing a clinical evaluation or for deriving a therapeutic intervention to perform, based on the results of the aforesaid predictive prognostic method.

Two examples of the information made available by the above method are shown in the tables of FIGS. 2 and 5.

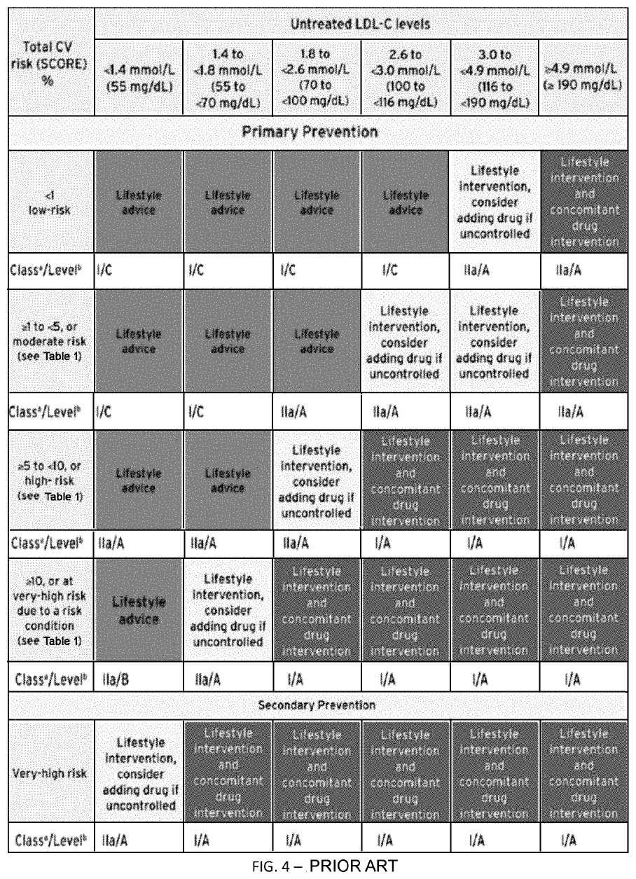

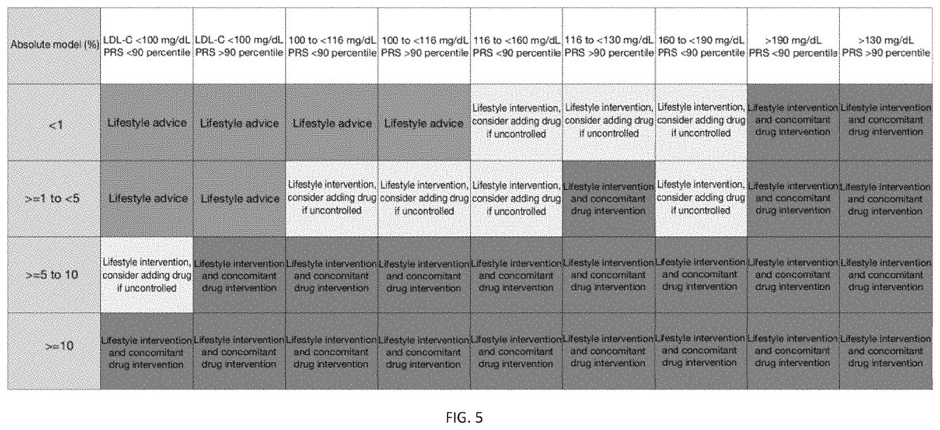

To better illustrate this aspect, the tables of FIG. 5 (i.e., a result of the present invention) and FIG. 4, which represents the related prior art, i.e., the current guidelines which indicate to the physician how to intervene based on a risk parameter of the individual (which represents non-genetic risk factors) and the LDL-C level of the individual, are compared below.

In the current guidelines, the LDL-C level is used as the decision maker for the intervention to be carried out on the patient. As shown in FIG. 4, 4 intervention levels are identified: “None”, “Lifestyle advice”, “Consider adding drugs if uncontrolled”, “Lifestyle intervention and concomitant drug intervention”.

The risk identified by means of the risk parameter (“score”) divides patients into 3 groups (rows) and the risk due to the LDL level into 5 columns. The intervention recommended for the patient can be identified at the intersection of each row and column.

However, such known methodology does not take into account the genetic risk factor, which can be represented by the PRS, which the principle forms as a further “dimension”, correlated with the previous ones, and which, according to an implementation option, can be included in a three-dimensional matrix replacing the known table of the guidelines.

Another implementation option, more advantageous for ease of use, is to consider the effect of the PRS through the definition of a “modified” LDL-C level which also takes into account the PRS value. Such a “modified” LDL-C level can be, for example, one of the aforementioned “individualized target value” VOI of LDL cholesterol level or “individualized equivalent value” VEI of LDL cholesterol level.

Such innovative methodology, included in the invention, is shown in FIG. 5, in which the columns are characterized by suitably modified LDL-C values which take into account how the PRS of the individual is positioned with respect to the population average.

For example, in the case of LDL-C equal to 100 (normal) in the presence of a high PRS, there could be a corresponding LDL equal to 120 (intermediate), and therefore, in the case of risk class between 5 and 10, it would shift from “consider adding drugs if uncontrolled” to “lifestyle intervention and concomitant drug intervention to lower LDL-C cholesterol”.

According to an embodiment, such a method comprises providing information on the therapeutic strategy to be followed based on a low-density lipoprotein cholesterol value LDL-C which is modified to take into account an individual's polygenic risk score PRS, and/or based on said “individualized target value” VOI of low-density lipoprotein cholesterol level, and/or based on said “individualized equivalent value” VEI of low-density lipoprotein cholesterol level.

As can be seen, the objects of the present invention, as previously indicated, are fully achieved by the method described above, by virtue of the features disclosed above in detail.

In fact, the method allows the genetic risk factor to be appropriately and effectively combined in combination with other risk factors, and is capable of providing improved results, with regard to reliability, accuracy and precision, especially regarding the correlation of the risk due to genetic factors and the risk linked to other factors such as the cholesterol level.

Furthermore, in addition to estimating the risk level, taking into account several factors including genetic ones, the present invention provides a method which, having as input the PRS of an individual (and in addition, possibly, other information about the additional risk factors already known), provides as a result a level (i.e., the aforementioned “individualized target value”) of LDL-C cholesterol to be achieved in order to obtain a risk equivalent to that of the average population. For example, it was found, by carrying out the method, that in the presence of a PRS in the 95th percentile with respect to the reference population, to have a risk equal to that of the average population, it is necessary to have 124 mg/dL of LDL-C.

In addition, the present invention provides a method which, having as input the LDL-C and PRS of an individual, provides as a result a level (the aforesaid “individualized equivalent value”) of LDL-C cholesterol which confers a risk equivalent to that conferred in an average population. For example, it was found, by carrying out the method, that with a PRS in the 95th percentile and an LDL-C of 140 mg/dL, there is the same risk as an average PRS and an LDL-C level equal to 200 mg/dL. This aspect of the present invention is particularly useful to a physician for diagnostic and therapeutic purposes.

In order to meet contingent needs, those skilled in the art may make modifications and adaptations to the embodiments of the method described above and can replace elements with others which are functionally equivalent without departing from the scope of the following claims. All the features described above as belonging to one possible embodiment may be implemented irrespective of the other embodiments described.

1. A method for a predictive prognosis of the onset of a cardiovascular disease in an individual, comprising the steps of:
calculating at least one risk value, representative of the risk of the onset of cardiovascular disease in the individual, based on an operational value of a polygenic risk score of the individual's and an operational value of the individual's low-density lipoprotein cholesterol level, and also based on a first risk parameter representative of a risk of onset of cardiovascular disease induced by the polygenic risk score, on a second risk parameter representative of a risk of onset of cardiovascular disease induced by the low-density lipoprotein cholesterol level, and on a third risk parameter representative of a risk of onset of cardiovascular disease induced by the interaction of polygenic risk score and low-density lipoprotein cholesterol level,
wherein said first risk parameter, second risk parameter and third risk parameter are calculated on the basis of a pre-trained model and/or algorithm, to which the operational value of polygenic risk score and operational value of low-density lipoprotein cholesterol level are provided in input;
based on said at least one calculated risk value, determining and providing at least one of the following prognostic results:
an individualized risk value of onset of cardiovascular disease in an individual to be examined; and/or
an individualized target value of low-density lipoprotein cholesterol level (LDL-C_{T}) such as to cause, for an individual having a given polygenic risk score, a risk corresponding to that of an individual having an average polygenic risk score value, and/or
an individualized equivalent value of low-density lipoprotein cholesterol level to cause, for an individual having a given polygenic risk score (PRSₚ) and a given individualized low-density lipoprotein cholesterol level, a risk corresponding to that of a sample individual having an average polygenic risk score value and the same individualized low-density lipoprotein cholesterol level value as the individual to be examined;
wherein the method further comprises the steps of accessing the genetic data of the individual to be examined, comprising the individual's single nucleotide polymorphisms, and calculating a polygenic risk score value of the individual, based on said genetic data of the individual; and wherein:
if the outcome of the prognosis is the individualized risk value (Rₚ):
the method comprises the further step of acquiring or accessing data comprising the individual's low-density lipoprotein cholesterol level;
the step of calculating at least one risk value comprises calculating a risk value considering as the operational value of polygenic risk score the value of the individual's polygenic risk score and as the operational value of the low-density lipoprotein cholesterol level the individual's low-density lipoprotein cholesterol level;
the individualized risk value of onset of cardiovascular disease corresponds to said calculated risk value;
if the outcome of the prognosis is the individualized target value of low-density lipoprotein cholesterol level (LDL-C_{T}):
the method comprises the further step of defining a plurality of sample values of low-density lipoprotein cholesterol level;
the step of calculating at least one risk value comprises, for each sample value of low-density lipoprotein cholesterol level, calculating a respective plurality of sample risk values (Rₙ) in which the polygenic risk score is the individual's polygenic risk score and a respective plurality of average risk values in which the polygenic risk score is a preset average polygenic risk score;
the step of determining an individualized target value of low-density lipoprotein cholesterol level comprises determining the individualized target value of low-density lipoprotein cholesterol level as the value of the plurality of sample values of low-density lipoprotein cholesterol level for which the respective sample risk value is equal to the respective average risk value;
if the result of the prognosis is the individualized equivalent value of the low-density lipoprotein cholesterol level:
the method comprises the further steps of acquiring or accessing data comprising the individual's low-density lipoprotein cholesterol level, and defining a plurality of sample low-density lipoprotein cholesterol level values;
the step of calculating at least one risk value comprises calculating an individualized risk value considering as the operational value of polygenic risk score the value (PRSₚ) of the individual's polygenic risk score and as the operational value of the low-density lipoprotein cholesterol level the individual's low-density lipoprotein cholesterol level;
the step of calculating at least one risk value further comprises calculating, for each sample value of low-density lipoprotein cholesterol level, a respective plurality of average risk values in which the polygenic risk score is a preset average polygenic risk score;
the step of determining an individualized equivalent value of low-density lipoprotein cholesterol level comprises determining the individualized equivalent value of low-density lipoprotein cholesterol level as the value of the plurality of sample values of low-density lipoprotein cholesterol level for which the respective average risk value is equal to the individual's individualized risk value.

2. A method according to claim 1, wherein said prognostic results comprise the individualized risk value of onset of cardiovascular disease in an individual to be examined and further at least one of said individualized target value of low-density lipoprotein cholesterol level and individualized equivalent value of low-density lipoprotein cholesterol level.

3. A method according to claim 1, wherein said prognostic results comprise the individualized target value of low-density lipoprotein cholesterol level and individualized equivalent value of low-density lipoprotein cholesterol level.

4. A method according to claim 1, wherein the pre-trained model and/or algorithm comprises an algorithm trained in a preliminary training step,
wherein the preliminary training step comprises training the algorithm by machine learning and/or artificial intelligence techniques, based on experimental data referring to individuals for which respective individual values of polygenic risk score and operational values of low-density lipoprotein cholesterol level are known and the onset or non-onset of cardiovascular disease is also known.

5. A method according to claim 4, wherein the pre-trained model and/or algorithm comprises a regression model, which uses as covariates the polygenic risk score, the low-density lipoprotein cholesterol level, the combination of polygenic risk score and the low-density lipoprotein cholesterol level,
and wherein the preliminary training step comprises training the regression model using as a reference dataset a first dataset of a population of individuals of which polygenic risk score and low-density lipoprotein cholesterol level are known, and for which the outcomes of onset of cardiovascular disease are known, for a first subset of individuals, or cases, or for which the non-onset of cardiovascular disease is known, after a predetermined period of time, for a second subset of individuals or controls.

6. A method according to claim 5, wherein the pre-trained regression model is used to derive said first risk parameter, second risk parameter and third risk parameter,
and wherein said preliminary training step comprises training the regression model with reference to said first risk parameter, second risk parameter and third risk parameter based on said reference dataset using as a dependent variable the onset or non-onset, obtainable from known data, of cardiovascular disease.

7. A method according to claim 5, wherein the pre-trained regression model is used to calculate said at least one risk value,
and wherein said preliminary training step comprises training the regression model with reference to said at least one risk value based on said reference dataset using as the dependent variable the onset or non-onset, obtainable from known data, of cardiovascular disease.

8. A method according to claim 5, wherein the regression model is a Cox proportional hazards regression model.

9. A method according to claim 1, adapted to perform a predictive prognosis which takes into account one or more further risk factors, wherein:
said step of calculating at least one risk value comprises calculating the at least one risk value also on the basis of an operational value of each of one or more further covariates associated with a respective one of said one or more further risk factors;
the pre-trained model and/or algorithm is configured to calculate said first risk parameter, second risk parameter and third risk parameter also on the basis of the operational values of each of said one or more further covariates,
and wherein the method further comprises accessing data of the individual to be examined, including individual values of each of said one or more further covariates associated with the one or more further risk factors,
and wherein:
as part of the determination of the individualized target value of low-density lipoprotein cholesterol level, the step of calculating at least one risk value comprises, for each sample value of low-density lipoprotein cholesterol level, calculating a respective plurality of sample risk values where the polygenic risk score is the individual's polygenic risk score and the value of each of the further covariates associated with a respective risk factor corresponds to, or is representative of, the individual value of the respective covariate associated with the risk factor found in the individual, and further comprises calculating a respective plurality of average risk values in which the polygenic risk score is a preset average polygenic risk score, the value of each of the further covariates associated with a respective risk factor corresponds to, or is representative of, the individual value of the respective covariate associated with the risk factor found in the individual;
as part of the determination of the individualized equivalent value of low-density lipoprotein cholesterol level, the step of calculating at least one risk value comprises:
calculating an individualized risk value considering as the operational value of polygenic risk score the value of the individual's polygenic risk score and as the operational value of the low-density lipoprotein cholesterol level the individual's low-density lipoprotein cholesterol level, and as the operational value of each of said further covariates associated with a respective risk factor a value corresponding to, representative of, the individual value of the respective covariate found in the individual;
calculating, for each sample value of low-density lipoprotein cholesterol level, a respective plurality of average risk values in which the polygenic risk score is an average polygenic risk score, and the value of each of the further covariates associated with a respective risk factor corresponds to, or is representative of, the individual value of the respective covariate associated with the risk factor found in the individual.

10. A method according to claim 9, wherein the pre-trained model and/or algorithm comprises a regression model, which uses as covariates, in addition to the polygenic risk score variable, the low-density lipoprotein cholesterol level variable, combination of polygenic risk score and low-density lipoprotein cholesterol level variable, also said further variables which encode the further risk factors,
and wherein the preliminary training step comprises training the regression model using as a reference dataset a second dataset of a population of individuals of which, in addition to polygenic risk score and low-density lipoprotein cholesterol level, also the individual values of each of said further covariates is known.

11. A method according to claim 1, wherein the individualized target value of low-density lipoprotein cholesterol level is determined, and wherein the method comprises:
determining in which percentile the individualized polygenic risk score falls within a distribution of polygenic risk score values of individuals of a known reference population;
performing a plurality of simulations, each using the pre-trained model and/or algorithm, wherein each simulation is performed on an individual with the same polygenic risk score value as the individual examined, and/or having the same values of covariates associated with other risk factors of the individual under examination, and on a respective sample value low-density lipoprotein cholesterol level, among a set of increasing values of low-density lipoprotein cholesterol level which vary from the minimum to the maximum of the values present in the reference population on which the pre-trained model was trained;
wherein each simulation determines a risk level corresponding to a respective sample value of low-density lipoprotein cholesterol level;
wherein each of the risk levels is compared with the risk calculated on an individual with the same features of the individual to be examined, but an average genetic risk, with an average polygenic risk score.

12. A method according to claim 1, wherein the individualized equivalent value of low-density lipoprotein cholesterol level is determined, and wherein the method comprises:
performing a plurality of simulations, each using the pre-trained model and/or algorithm, wherein each simulation is performed on an individual having the same values of covariates associated with other risk factors as the individual in question, but having an average polygenic risk score value, and on a respective sample value of low-density lipoprotein cholesterol level, among a set of increasing values of low-density lipoprotein cholesterol level which vary from the minimum to the maximum of the values present in the reference population on which the pre-trained model was trained;
wherein each simulation determines a level of risk corresponding to a respective sample value of low-density lipoprotein cholesterol level;
to obtain the low-density lipoprotein cholesterol level which confers the same final risk of developing cardiovascular disease for an individual with the same features as the individual to be examined, but an average polygenic risk score.

13. A method according to claim 1, wherein the risk value is calculated based on a sum of products related to different variables, comprising at least said polygenic risk score, low-density lipoprotein cholesterol level, combination of polygenic risk score and low-density lipoprotein cholesterol level, wherein each product involves the multiplication of said first risk parameter, second risk parameter and third risk parameter for the difference between the operational value and the average value of the variables, respectively, polygenic risk score, low-density lipoprotein cholesterol level, combination of polygenic risk score and low-density lipoprotein cholesterol level.

14. A method according to claim 1, wherein the determined or calculated risk value is a relative risk value, with respect to an average risk referring to a population,
or wherein the determined or calculated risk value is an absolute risk value, related to the individual, and representative of an individual's risk of developing cardiovascular disease within a certain period of time, or at a given age.

15. A method according to claim 1, wherein the step of calculating a value of the individual's polygenic risk score, based on the individual's genetic data, comprises:
identifying, in the individual's genetic data, a personalized subset of single nucleotide polymorphisms which also belong to a set, wherein each of the single nucleotide polymorphisms of said predetermined set comprises a single nucleotide polymorphism identifier, and is associated with a respective relevance parameter;
calculating the individual's polygenic risk score value, based on said personalized subset (SI₁) of single nucleotide polymorphisms and the respective relevance parameters (βᵢ),
wherein said single nucleotide polymorphism identifier comprises a genetic variant address and an active allele present in said genetic variant address; said personalized subset (SI₁) comprises single nucleotide polymorphisms in which said active allele is traced as present in the respective genetic variant address, in the individual's genetic data, and is associated with a respective allelic dosage; said step of calculating a value of individualized polygenic risk score comprises summing the values of all the first relevance parameters associated with all the respective single nucleotide polymorphisms of the customized subset, each multiplied by the respective allelic dosage of the active allele;
and wherein the determination of said predetermined set of single nucleotide polymorphisms and the calculation of said relevance parameters are performed in a preliminary training step, comprising the training of at least one algorithm using machine learning and/or artificial intelligence techniques, based on known data, or in which said predetermined set of single nucleotide polymorphisms and said relevance parameters are taken from known tables.

16. A method according to claim 1, wherein the cardiovascular disease is a coronary artery disease.

17. A device adapted to perform a predictive prognosis of the onset of a cardiovascular disease, comprising:
acquisition means for acquiring an amount of blood of an individual and determining the individual's low-density lipoprotein cholesterol level based on a test of the amount of blood acquired;
an electronic interface, adapted to receive in input a calculated value of the individual's polygenic risk score;
a processor, configured to receive said low-density lipoprotein cholesterol level and polygenic risk score value of the individual, and to perform a method for a predictive prognosis of the onset of cardiovascular disease;
wherein said acquisition and determination means, said electronic interface and said processor are in a single portable device;
wherein said method for a predictive prognosis of the onset of a cardiovascular disease comprises the steps of:
calculating at least one risk value, representative of the risk of the onset of cardiovascular disease in an individual, based on an operational value of a polygenic risk score of the individual's and an operational value of the individual's low-density lipoprotein cholesterol level, and also based on a first risk parameter representative of a risk of onset of cardiovascular disease induced by the polygenic risk score, on a second risk parameter representative of a risk of onset of cardiovascular disease induced by the low-density lipoprotein cholesterol level, and on a third risk parameter representative of a risk of onset of cardiovascular disease induced by the interaction of polygenic risk score and low-density lipoprotein cholesterol level,
wherein said first risk parameter, second risk parameter and third risk parameter are calculated on the basis of a pre-trained model and/or algorithm, to which the operational value of polygenic risk score and operational value of low-density lipoprotein cholesterol level are provided in input;
based on said at least one calculated risk value, determining and providing at least one of the following prognostic results:
an individualized risk value of onset of cardiovascular disease in an individual to be examined; and/or
an individualized target value of low-density lipoprotein cholesterol level to cause, for an individual having a given polygenic risk score, a risk corresponding to that of an individual having an average polygenic risk score value, and/or
an individualized equivalent value of low-density lipoprotein cholesterol level to cause, for an individual having a given polygenic risk score and a given individualized low-density lipoprotein cholesterol level, a risk corresponding to that of a sample individual having an average polygenic risk score value and the same individualized low-density lipoprotein cholesterol level value as the individual to be examined;
accessing the genetic data of the individual to be examined, comprising the individual's single nucleotide polymorphisms, and calculating a polygenic risk score value of the individual, based on said genetic data of the individual; and wherein:
if the outcome of the prognosis is the individualized risk value:
the method comprises the further step of acquiring or accessing data comprising the individual's low-density lipoprotein cholesterol level;
the step of calculating at least one risk value comprises calculating a risk value considering as the operational value of polygenic risk score the value of the individual's polygenic risk score and as the operational value of the low-density lipoprotein cholesterol level the individual's low-density lipoprotein cholesterol level;
the individualized risk value of onset of cardiovascular disease corresponds to said calculated risk value;
if the outcome of the prognosis is the individualized target value of low-density lipoprotein cholesterol level:
the method comprises the further step of defining a plurality of sample values of low-density lipoprotein cholesterol level;
the step of calculating at least one risk value comprises, for each sample value of low-density lipoprotein cholesterol level, calculating a respective plurality of sample risk values in which the polygenic risk score is the individual's polygenic risk score and a respective plurality of average risk values in which the polygenic risk score is a preset average polygenic risk score;
the step of determining an individualized target value of low-density lipoprotein cholesterol level comprises determining the individualized target value of low-density lipoprotein cholesterol level as the value of the plurality of sample values of low-density lipoprotein cholesterol level for which the respective sample risk value is equal to the respective average risk value;
if the result of the prognosis is the individualized equivalent value of the low-density lipoprotein cholesterol level:
the method comprises the further steps of acquiring or accessing data comprising the individual's low-density lipoprotein cholesterol level, and defining a plurality of sample low-density lipoprotein cholesterol level values;
the step of calculating at least one risk value comprises calculating an individualized risk value considering as the operational value of polygenic risk score the value of the individual's polygenic risk score and as the operational value of the low-density lipoprotein cholesterol level the individual's low-density lipoprotein cholesterol level;
the step of calculating at least one risk value further comprises calculating, for each sample value of low-density lipoprotein cholesterol level, a respective plurality of average risk values in which the polygenic risk score is a preset average polygenic risk score;
the step of determining an individualized equivalent value of low-density lipoprotein cholesterol level comprises determining the individualized equivalent value of low-density lipoprotein cholesterol level as the value of the plurality of sample values of low-density lipoprotein cholesterol level for which the respective average risk value is equal to the individual's individualized risk value.

18. A method for providing a clinical evaluation or for deriving a therapeutic intervention, wherein said method is performed based on the results of a method for a predictive prognosis of the onset of a cardiovascular disease comprising the steps of:
calculating at least one risk value, representative of the risk of the onset of cardiovascular disease in an individual, based on an operational value of a polygenic risk score of the individual's and an operational value of the individual's low-density lipoprotein cholesterol level, and also based on a first risk parameter representative of a risk of onset of cardiovascular disease induced by the polygenic risk score, on a second risk parameter representative of a risk of onset of cardiovascular disease induced by the low-density lipoprotein cholesterol level, and on a third risk parameter representative of a risk of onset of cardiovascular disease induced by the interaction of polygenic risk score and low-density lipoprotein cholesterol level,
wherein said first risk parameter, second risk parameter and third risk parameter are calculated on the basis of a pre-trained model and/or algorithm, to which the operational value of polygenic risk score and operational value of low-density lipoprotein cholesterol level are provided in input;
based on said at least one calculated risk value, determining and providing at least one of the following prognostic results:
an individualized risk value of onset of cardiovascular disease in an individual to be examined; and/or
an individualized target value of low-density lipoprotein cholesterol level to cause, for an individual having a given polygenic risk score, a risk corresponding to that of an individual having an average polygenic risk score value, and/or
an individualized equivalent value of low-density lipoprotein cholesterol level to cause, for an individual having a given polygenic risk score and a given individualized low-density lipoprotein cholesterol level, a risk corresponding to that of a sample individual having an average polygenic risk score value and the same individualized low-density lipoprotein cholesterol level value as the individual to be examined;
wherein the method further comprises the steps of accessing the genetic data of the individual to be examined, comprising the individual's single nucleotide polymorphisms, and calculating a polygenic risk score value of the individual, based on said genetic data of the individual; and
wherein:
if the outcome of the prognosis is the individualized risk value:
the method comprises the further step of acquiring or accessing data comprising the individual's low-density lipoprotein cholesterol level;
the step of calculating at least one risk value comprises calculating a risk value considering as the operational value of polygenic risk score the value of the individual's polygenic risk score and as the operational value of the low-density lipoprotein cholesterol level the individual's low-density lipoprotein cholesterol level;
the individualized risk value of onset of cardiovascular disease corresponds to said calculated risk value;
if the outcome of the prognosis is the individualized target value of low-density lipoprotein cholesterol level:
the method comprises the further step of defining a plurality of sample values of low-density lipoprotein cholesterol level;
the step of calculating at least one risk value comprises, for each sample value of low-density lipoprotein cholesterol level, calculating a respective plurality of sample risk values in which the polygenic risk score is the individual's polygenic risk score and a respective plurality of average risk values in which the polygenic risk score is a preset average polygenic risk score;
the step of determining an individualized target value of low-density lipoprotein cholesterol level comprises determining the individualized target value of low-density lipoprotein cholesterol level as the value of the plurality of sample values of low-density lipoprotein cholesterol level for which the respective sample risk value is equal to the respective average risk value;
if the result of the prognosis is the individualized equivalent value of the low-density lipoprotein cholesterol level:
the method comprises the further steps of acquiring or accessing data comprising the individual's low-density lipoprotein cholesterol level, and defining a plurality of sample low-density lipoprotein cholesterol level values;
the step of calculating at least one risk value comprises calculating an individualized risk value considering as the operational value of polygenic risk score the value of the individual's polygenic risk score and as the operational value of the low-density lipoprotein cholesterol level the individual's low-density lipoprotein cholesterol level;
the step of calculating at least one risk value further comprises calculating, for each sample value of low-density lipoprotein cholesterol level, a respective plurality of average risk values in which the polygenic risk score is a preset average polygenic risk score;
the step of determining an individualized equivalent value of low-density lipoprotein cholesterol level comprises determining the individualized equivalent value of low-density lipoprotein cholesterol level as the value of the plurality of sample values of low-density lipoprotein cholesterol level for which the respective average risk value is equal to the individual's individualized risk value.

19. A method according to claim 18, wherein the method comprises:
providing information on the therapeutic strategy to be followed based on a low-density lipoprotein cholesterol value which is modified to take into account an individual's polygenic risk score, and/or based on said individualized target value of low-density lipoprotein cholesterol level, and/or based on said individualized equivalent value of low-density lipoprotein cholesterol level.
